(19) United States
(12) Patent Application Publication
(10) Pub. No.: US 2020/0297601 A1
(43) Pub. Date: Sep. 24, 2020

(54) COMPOSITION FOR SKIN MOISTURIZATION OR SKIN WHITENING, CONTAINING PENTACYCLIC TRITERPENE CAFFEIC ACID ESTERS

(71) Applicant: AMOREPACIFIC CORPORATION, Seoul (KR)

(72) Inventors: Sejin YOO, Yongin-si, Gyeonggi-do (KR); Nok Hyun PARK, Yongin-si, Gyeonggi-do (KR); Jeong AH, Yongin-si, Gyeonggi-do (KR); Hyunwoo LEE, Yongin-si, Gyeonggi-do (KR); Yongjin KIM, Yongin-si, Gyeonggi-do (KR)

(21) Appl. No.: 16/089,161

(22) PCT Filed: Mar. 29, 2017
(86) PCT No.: PCT/KR2017/003427

§ 371 Date: Sep. 27, 2018

(30) Foreign Application Priority Data
Mar. 31, 2016 (KR) 10-2016-0039509

(51) Int. Cl. A61K 8/37; A61K 8/42; A61Q 19/00; A61Q 19/02

(52) U.S. Cl. CPC A61K 8/375; A61Q 19/02; A61Q 19/007; A61K 8/42

(57) ABSTRACT

The present specification relates to a composition for skin moisturizing or skin whitening, containing, as an active ingredient: 3β,23,28-trihydroxy-12-oleanene 23-caffeate, a salt thereof, an isomer thereof, a hydrate thereof or a solvate thereof; and/or 3β,23,28-trihydroxy-12-oleanene 3β-caffeate, an isomer thereof, a hydrate thereof or a solvate thereof, wherein the composition can be used as a composition for external application to the skin or as a cosmetic composition, and the composition has a skin moisturization or skin whitening effect.

### TECHNICAL FIELD

The present disclosure relates to a composition for skin moisturizing or skin whitening, which contains a pentacyclic triterpene caffeic acid ester as an active ingredient.

### BACKGROUND ART

It is known that a hibisci cortex extract has anti-aging, skin whitening and skin moisturizing effects. For pentacyclic triterpene caffeic acid esters contained in the hibisci cortex extract, the effect of inhibiting lipid peroxidation and the toxic effect on cancer cells are known. However, nothing is known about the skin moisturizing or skin whitening effect of the pentacyclic triterpene caffeic acid esters when they are used, for example, in cosmetic compositions.

The inventors of the present disclosure have studied on the skin moisturizing and skin whitening effects of the pentacyclic triterpene caffeic acid esters and have completed the present disclosure.

### REFERENCES OF RELATED ART

### Patent Documents

Korean Patent Publication No. 10-2010-0059302.

Korean Patent Publication No. 10-2001-0010240.

Chinese Patent Publication No. CN103342730A.

Japanese Patent Publication No. 2007-051101.

### DISCLOSURE

### Technical Problem

The inventors of the present disclosure have studied on pentacyclic triterpene caffeic acid esters and have found out that the pentacyclic triterpene caffeic acid esters have skin moisturizing and skin whitening effects.

In an aspect, the present disclosure is directed to providing a composition for skin moisturizing or skin whitening, which contains a pentacyclic triterpene caffeic acid ester as an active ingredient.

### Technical Solution

In an aspect, the present disclosure provides a composition for skin moisturizing, which contains one or more of 3β3,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof, as a pentacyclic triterpene caffeic acid ester, as an active ingredient.

In an aspect, the present disclosure provides a composition for skin whitening, which contains one or more of 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof, as a pentacyclic triterpene caffeic acid ester, as an active ingredient.

In an aspect of the present disclosure, the active ingredient is 3β,23,28-trihydroxy-12-oleanene 23-caffeate, a salt thereof, an isomer thereof, a hydrate thereof or a solvate thereof.

In an aspect of the present disclosure, the active ingredient is 3β,23,28-trihydroxy-12-oleanene 3β-caffeate, a salt thereof, an isomer thereof, a hydrate thereof or a solvate thereof.

In an aspect of the present disclosure, the active ingredient is contained in an amount of 0.0001-1 wt % based on the total weight of the composition.

In an aspect of the present disclosure, the composition contains 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof.

In an aspect of the present disclosure, the composition contains 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof at a weight ratio of 1:0.5-1.5.

In an aspect of the present disclosure, the composition for skin moisturizing further contains a ceramide.

In an aspect of the present disclosure, the composition is a composition for external application to skin.

In an aspect of the present disclosure, the composition is a cosmetic composition.

### Advantageous Effects

A composition according to an aspect of the present disclosure has a superior skin moisturizing effect.

The composition according to an aspect of the present disclosure has a superior skin whitening effect.

The composition according to an aspect of the present disclosure has a superior effect of promoting expression of hyaluronic acid synthase when applied to skin.

The composition according to an aspect of the present disclosure has a superior effect of promoting expression of caspase 14 when applied to skin.

The composition according to an aspect of the present disclosure has a superior effect of reducing melanin content when applied to skin.

### BRIEF DESCRIPTION OF DRAWINGS

FIG. 1 and FIG. 2 show results of evaluating expression of HAS and caspase 14 in Test Example 1 of the present disclosure.

FIG. 3 shows immunofluorescence microscopic images obtained in Test Example 2 of the present disclosure.

FIG. 4 shows a result of evaluating skin whitening effect in Test Example 3 of the present disclosure.

### BEST MODE

Hereinafter, the present disclosure is described in detail.

In an aspect, the present disclosure provides a composition for skin moisturizing, which contains one or more of 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof, as a pentacyclic triterpene caffeic acid ester, as an active ingredient.

In an aspect, the present disclosure provides a composition for skin whitening, which contains one or more of 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof, as a pentacyclic triterpene caffeic acid ester, as an active ingredient.

In an aspect of the present disclosure, the 3β,23,28-trihydroxy-12-oleanene 23-caffeate may be represented by Chemical Formula 1. The compound of Chemical Formula 1 has a CAS No. 226406-74-2.

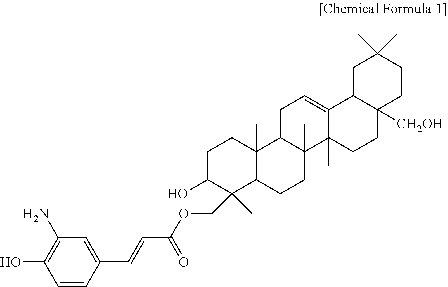

In an aspect of the present disclosure, the 3β,23,28-trihydroxy-12-oleanene 3β-caffeate may be represented by Chemical Formula 2. The compound of Chemical Formula 2 has a CAS No. 226406-72-0.

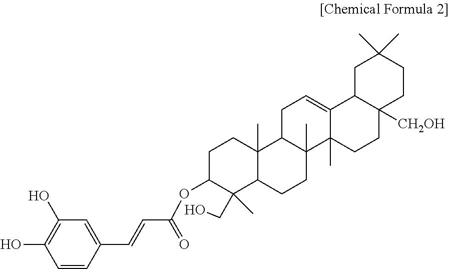

In an aspect of the present disclosure, the 3β,23,28-trihydroxy-12-oleanene 23-caffeate may be represented by Chemical Formula 3, wherein R¹ is —OH and R² is represented by Chemical Formula 4.

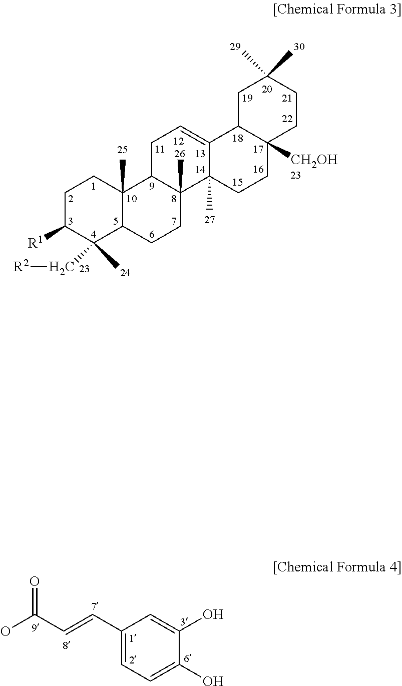

In an aspect of the present disclosure, in the 3β,23,28-trihydroxy-12-oleanene 3β-caffeate represented by Chemical Formula 3, R¹ may be represented by Chemical Formula 4 and R² may be —OH.

In the present disclosure, the 3β,23,28-trihydroxy-12-oleanene 23-caffeate of Chemical Formula 1 or the 3β,23,28-trihydroxy-12-oleanene 3β-caffeate of Chemical Formula 2 may be derived from hibisci cortex. In the present disclosure, the “hibisci cortex” refers to the dried root bark or stem bark of rose of Sharon (Hibiscus syriacus L.), which is a deciduous shrub in the family Malvaceae. The root bark or stem bark is peeled off and dried under the sun in spring. It tastes sweet and bitter and has a neutral characteristic. It acts on the liver, spleen, large intestine and small intestine. It clears away heat and toxins, eliminates dampness and stops itching. In addition, it helps blood circulation and stops bleeding. It is used for enterohemorrhage, dysentery, rectal prolapse, leucorrhea, scabies, athlete's foot, hemorrhoid, etc. 3-9 g a day is taken as an infusion or applied externally after being boiled down with water or brewed in liquor.

In the present disclosure, the “isomer” includes not only optical isomers (e.g., essentially pure enantiomers, essentially pure diastereomers or mixtures thereof) but also conformational isomers (i.e., isomers differing only in angles of one or more chemical bonds), constitutional isomers (especially, tautomers) or geometric isomers (e.g., cis-trans isomers).

In the present disclosure, “essentially pure” means, for example, when used in connection with enantiomers or diastereomers, that the specific compound as an example of the enantiomer or the diastereomer is present in an amount of about 90% (w/w) or more, specifically about 95% or more, more specifically about 97% or more or about 98% or more, further more specifically about 99% or more, even more specifically about 99.5% or more.

In the present disclosure, the “hydrate” refers to a compound bound with water. It is used in a broad sense, including an inclusion compound which lacks chemical bonding between water and the compound.

In the present disclosure, the “solvate” refers to a higher-order compound formed between a solute molecule or ion and a solvent molecule or ion.

In an aspect of the present disclosure, the active ingredient may be 3β,23,28-trihydroxy-12-oleanene 23-caffeate, a salt thereof, an isomer thereof, a hydrate thereof or a solvate thereof.

In an aspect of the present disclosure, the active ingredient may be 3β,23,28-trihydroxy-12-oleanene 3β-caffeate, a salt thereof, an isomer thereof, a hydrate thereof or a solvate thereof.

In an aspect of the present disclosure, the active ingredient may be contained in an amount of 0.0001-1 wt % based on the total weight of the composition. Specifically, the composition for skin moisturizing or the composition for skin whitening may contain the active ingredient in an amount of 0.0001 wt % or more, 0.0003 wt % or more, 0.0004 wt % or more, 0.0005 wt % or more, 0.0006 wt % or more, 0.0007 wt % or more, 0.0008 wt % or more, 0.001 wt % or more, 0.0012 wt % or more, 0.0015 wt % or more, 0.002 wt % or more, 0.005 wt % or more, 0.01 wt % or more, 0.05 wt % or more, 0.1 wt % or more, 0.2 wt % or more, 0.3 wt % or more, 0.4 wt % or more, 0.5 wt % or more or 0.7 wt % or more based on the total weight of the composition. And, the composition for skin moisturizing or the composition for skin whitening may contain the active ingredient in an amount of 1 wt % or less, 0.5 wt % or less, 0.1 wt % or less, 0.05 wt % or less, 0.01 wt % or less, 0.005 wt % or less, 0.004 wt % or less, 0.003 wt % or less, 0.002 wt % or less, 0.001 wt % or less, 0.0008 wt % or less or 0.0005 wt % or less based on the total weight of the composition. A superior skin moisturizing or skin whitening effect is obtained when the content of the active ingredient is within the above-described range.

In an aspect of the present disclosure, the composition may contain 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof.

In an aspect of the present disclosure, the 3β,23,28-trihydroxy-12-oleanene 23-caffeate and the 3β,23,28-trihydroxy-12-oleanene 3β-caffeate may be contained at a weight ratio of 1:0.5-1.5. Specifically, the 3β,23,28-trihydroxy-12-oleanene 23-caffeate and the 3β,23,28-trihydroxy-12-oleanene 3β-caffeate may be contained at a weight ratio of 1:0.6-1.4, 1:0.8-1.3, 1:0.9-1.2, 1:0.9-1.1 or 1:1. A superior skin moisturizing or skin whitening effect is obtained when the weight ratio of the 3β,23,28-trihydroxy-12-oleanene 23-caffeate and the 3β,23,28-trihydroxy-12-oleanene 3β-caffeate is within the above-described range.

In an aspect of the present disclosure, the composition for skin moisturizing may further contain a ceramide.

In the present disclosure, the ceramide may be one or more selected from a group consisting of a natural ceramide and a pseudoceramide. The natural ceramide is a naturally occurring ceramide and includes ceramide 1, ceramide 2, ceramide 3, ceramide 4, ceramide 5, ceramide 6, ceramide 7, ceramide 8, etc. The pseudoceramide includes ceramide 104, ceramide 102, etc.

In the present disclosure, the pseudoceramide collectively refers to a compound having the lamellar structure of the natural ceramide.

In an aspect of the present disclosure, the pentacyclic triterpene caffeic acid ester as the active ingredient and the ceramide may be contained at a weight ratio of 1:0.1-1.5. Specifically, the weight ratio of the pentacyclic triterpene caffeic acid ester and the ceramide may be 1:0.15-1, 1:0.18-0.9, 1:0.19-0.8, 1:0.2-0.5 or 1:0.2. A superior skin moisturizing or skin whitening effect is obtained when the weight ratio of the pentacyclic triterpene caffeic acid ester and the ceramide is within the above-described range.

In an aspect of the present disclosure, the composition may be a composition for external application to skin. The composition for external application to skin may be used as a pharmaceutical composition for treating skin dryness or whitening skin. The composition for external application to skin is a general term including any composition that can be applied from outside of skin and cosmetics and medicines of various formulations can be included therein. The composition for external application to skin may be, for example, a composition for external application to skin for skin moisturizing or skin whitening, although not being particularly limited thereto.

In an aspect of the present disclosure, the composition may be a cosmetic composition.

The formulation of the cosmetic composition is not particularly limited and may be selected adequately depending on purposes. For example, it may be prepared into one or more formulation selected from a group consisting of a softening lotion (skin lotion or milk lotion), a nourishing lotion, an essence, a nourishing cream, a massage cream, a pack, a gel, an eye cream, an eye essence, a cleansing cream, a cleansing foam, a cleansing water, a powder, a body lotion, a body cream, a body oil and a body essence, although not being limited thereto. The content of the active ingredient is not particularly limited but may be 0.0001-10 wt % based on the total weight of the composition. A superior effect may be achieved without side effects when the content of the active ingredient satisfies the above-described condition. The cosmetic composition may further contain a cosmetically acceptable excipient serving as a diluent, a dispersant or a carrier such that the composition can be uniformly applied on skin. Specifically, the composition may be an oil-in-water (O/W) emulsion and the emulsion may contain at least 80 wt % of water as an excipient. However, without being limited thereto, any known cosmetically acceptable excipient may also be used. In addition, the cosmetic composition may contain various cosmetic adjuvants used in the art such as a fatty substance, an organic solvent, silicon, a thickener, an emollient, a sunscreen agent, an antifoaming agent, a moisturizing agent, a fragrance, a preservative, a surfactant, a filler, a sequestrant, a cationic, anionic, nonionic or amphoteric polymer or a mixture thereof, a propellant, an alkalizing or acidifying agent, a dye, a pigment or a nanopigment (especially, one prepared to supplementally provide sunscreen effect by physically blocking UV radiation) or other ingredients commonly used in cosmetics, especially in sunscreen compositions. The organic solvent may be a lower alcohol or a polyol, e.g., ethanol, isopropanol, propylene glycol, glycerol, sorbitol, etc. The fatty substance may be an oil, a wax or a mixture thereof, a fatty acid, a fatty acid ester, a fatty alcohol, petrolatum, paraffin, lanolin, hydrogenated lanolin or acetylated lanolin. The oil may be a plant, mineral or synthetic oil and may be selected, especially, from hydrogenated palm oil, hydrogenated castor oil, liquid petrolatum, liquid paraffin, purcellin oil, volatile or nonvolatile silicone oil and isoparaffin.

Hereinafter, the present disclosure will be described in detail through examples. However, the following examples are for illustrative purposes only and the scope of the present disclosure is not limited by the examples.

### Examples 1-4 and Comparative Example 1

A hibisci cortex extract obtained from hibisci cortex purchased from Kunwha Pharmaceutical was used for Comparative Example 1.

3β,23,28-Trihydroxy-12-oleanene 23-caffeate (PTCE 1) was used for Example 1 and 3β,23,28-trihydroxy-12-oleanene 3β-caffeate (PTCE 2) was used for Example 2.

### Extraction Method of Comparative Example 1

A. Dried hibisci cortex was extracted with 70% ethanol at 50° C. for 3 hours.

B. The extract was separated using a 1.2 μm filter and used for Comparative Example 1 (hibisci cortex extract: HE).

Skin moisturizing and skin whitening tests were conducted using the substances of Examples 1-2 according to Table 1.

TABLE 1

| | |
|---|---|
| Example 1 | 3β,23,28-Trihydroxy-12-oleanene 23-caffeate, 10 ppm |
| Example 2 | 3β,23,28-Trihydroxy-12-oleanene 3β-caffeate, 10 ppm |
| Example 3 | 5 ppm Example 1 + 5 ppm Example 2 |
| Example 4 | 10 ppm Example 1 + 2 ppm ceramide PC 104 (Macrocare) |

### Test Example 1: Evaluation of Skin Moisturization (Quantification of Hyaluronic Acid Synthase 2 (HAS2) and Caspase 14 by Real-Time PCR)

HAS2 (hyaluronan synthase 2) is a gene that produces hyaluronan or hyaluronic acid (HA). Caspase 14 is a protein which is involved in keratinocyte terminal differentiation and is important for the formation of the skin barrier. In skin moisturization, HA fills the skin and caspase 14 prevents water loss by strengthening the skin barrier. It is also involved degradation of filaggrin and production of natural moisturizing factors (NMFs). The expression level of HAS2 and caspase 14 was investigated for Comparative Example 1 and Examples 1-3.

A. Cell Culturing

Keratinocyte HaCaT cells were cultured in a DMEM medium containing 10% FBS and streptomycin in a 5% CO₂ incubator at 37° C. The cells were subcultured after the confluency reached about 90%.

B. Real-Time PCR

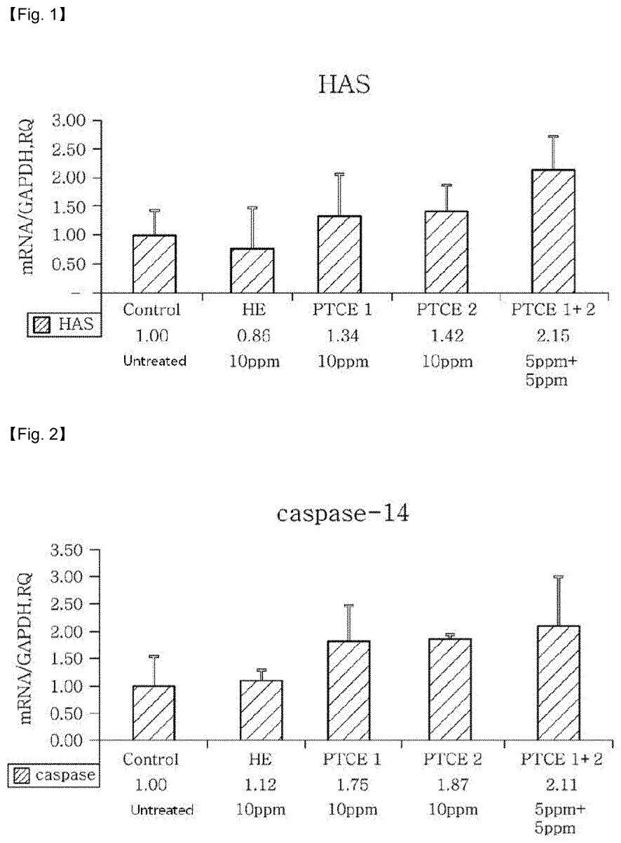

The HaCat cells cultured in A were seeded onto a 12-well plate with 1×10⁴ cells per well. After incubation for 24 hours, the cells were treated with Examples 1-3 and Comparative Example 1. 24 hours later, the cells were harvested for extraction of RNA. The RNA prepped using Invitrogen's TRIzol was subjected to RT-PCR using Invitrogen's Superscript Ill. Real-time qPCR was conducted using the obtained cDNA and the Taqman probe. Relative mRNA level was analyzed by measuring the change of fluorescence form SYBR Green I using the ICycler software. HAS2 and caspase 14 primers available from Life technologies were used (HAS2: Hs00193435_m1, caspase14: Hs00201637_m1). The quantitative expression level of the genes was corrected using GAPDH (glyceraldehyde 3-phosphate dehydrogenase) as an internal standard. The expression level of the HAS and caspase 14 genes is shown in FIGS. 1 and 2, respectively.

As seen from FIG. 1 and FIG. 2, the groups treated with Examples 1-3 showed superior skin moisturizing effect because the expression level of the HAS and caspase 14 genes was higher as compared to the untreated group. Especially, Example 3 showed the highest expression level of the HAS and caspase 14 genes. In addition, the groups treated with Examples 1-3 showed remarkably increased expression of the HAS and caspase 14 genes as compared to the hibisci cortex extract (HE) of Comparative Example 1.

### Test Example 2: Evaluation of Skin Moisturization

The skin moisturizing effect of Example 1 and Example 4 was evaluated by immunofluorescent staining and imaging of filaggrin which is known as an important skin moisturizing factor.

The surface of artificial skin (AmoreReskin™) was treated with 2 ppm ceramide PC 104, Example 1 and Example 4 for 4 days and then immunofluorescent staining was conducted. The immunofluorescent staining was conducted as follows.

Method of the Immunofluorescent Staining

i. Artificial skin is immersed in OCT solution (optimal cutting temperature, Tissue Tek) and stored at −70° C.

ii. The artificial skin is prepared into 10-μm thick sections using a cryotome and fixed on a slide.

iii. Lines are drawn around the artificial skin sections using a PAP pen.

iv. The slide is dried at room temperature for at least 8 hours.

v. The artificial skin sections are washed with PBS and dissolved in OCT solution.

vi. The artificial skin sections are treated with a blocking solution (1% BSA in PBS) for 30 minutes.

vii. The sections are treated with anti-filaggrin antibody (ab24584, Abcam) for 2 hours.

viii. The sections are treated with Texas Red anti-rabbit antibody (T6391, Life technologies) diluted to 1:200 in a blocking solution for 1 hour.

ix. The sections are stained with DAPI (4′,6-diamidino-2-phenylindole) for 5 minutes.

x. The sections are washed with PBS 3 times.

xi. After removing PBS, adding a mounting solution and covering a cover glass, the artificial skin sections are solidified.

xii. The sections are observed under a LSM510 microscope.

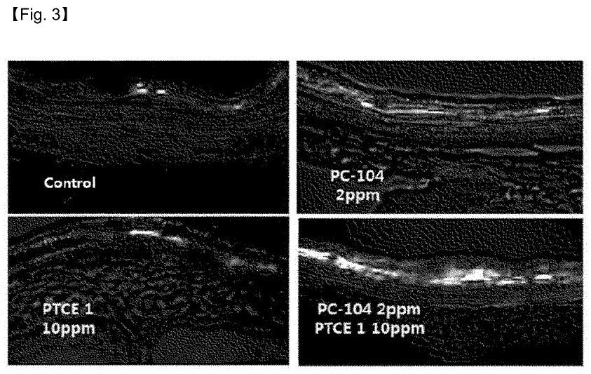

The result is shown in FIG. 3. In the original images of FIG. 3, the filaggrin protein was shown blue. In the grayscale images of FIG. 3, the blue regions in the original images were converted to white regions. Accordingly, the filaggrin protein is shown white in the grayscale images of FIG. 3.

As seen from FIG. 3, the filaggrin protein shown white was significantly increased for Examples 1 and 4. Accordingly, it was confirmed that skin moisturizing effect was improved because the filaggrin protein was increased for Examples 1 and 4.

### Test Example 3: Evaluation of Skin Whitening (Melanin Assay Using B16 Melanoma Cells)

B16 melanoma cells were seeded onto a 12-well plate with 1.5×10⁵ cells per well and then cultured for 24 hours. After treating with Examples 1-3, Comparative Example 1 or a positive control, the cells were cultured for 6 days while exchanging with a fresh medium once in three days. After the treatment with the test substances, the cells were washed with DPBS and then lysed at 60° C. for 1 hour after adding 2 N NaOH (10% DMSO). The cell culture and the cell lysate were transferred to a 96-well plate and absorbance was measured at 475 nm. Then, melanin content was calculated from a standard curve for synthetic melanin. The calculated melanin content was normalized to the total protein quantity for each test group and then compared with that of the control group.

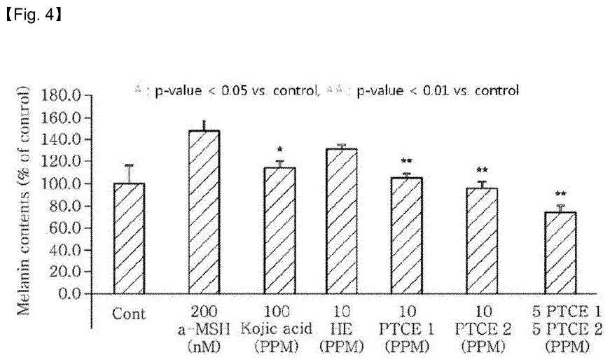

The result is shown in FIG. 4. As seen from FIG. 4, Examples 1-3 showed superior skin whitening effect as compared to Comparative Example 1 and the positive control group.

Hereinafter, the present disclosure will be described in detail through formulation examples. However, they are for illustrative purposes only and the scope of the present disclosure is not limited thereby.

### [Formulation Example 1] Softening Lotion (Skin Lotion)

TABLE 2

| Ingredients | Contents (wt %) |
|---|---|
| Example 1, Example 2 or Example 3 | 0.001 |
| Glycerin | 3.0 |
| Butylene glycol | 2.0 |
| Propylene glycol | 2.0 |
| Carboxyvinyl polymer | 0.1 |
| PEG-12 nonyl phenyl ether | 0.2 |
| Polysorbate 80 | 0.4 |
| Ethanol | 10.0 |
| Triethanolamine | 0.1 |
| Antiseptic, pigment and fragrance | Adequate |
| Purified water | Balance |

### [Formulation Example 2] Nourishing Lotion (Milk Lotion)

TABLE 3

| Ingredients | Contents (wt %) |
|---|---|
| Example 1, Example 2 or Example 3 | 0.001 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Antiseptic, pigment and fragrance | Adequate |
| Purified water | Balance |

### [Formulation Example 3] Nourishing Cream

TABLE 4

| Ingredients | Contents (wt %) |
|---|---|
| Example 1, Example 2 or Example 3 | 0.001 |
| Glycerin | 3.0 |
| Butylene glycol | 3.0 |
| Liquid paraffin | 7.0 |
| β-Glucan | 7.0 |
| Carbomer | 0.1 |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Triethanolamine | 0.1 |
| Antiseptic, pigment and fragrance | Adequate |
| Purified water | Balance |

### [Formulation Example 4] Massage Cream

TABLE 5

| Ingredients | Contents (wt %) |
|---|---|
| Example 1, Example 2 or Example 3 | 0.001 |
| Glycerin | 8.0 |
| Butylene glycol | 4.0 |
| Liquid paraffin | 45.0 |
| β-Glucan | 7.0 |
| Carbomer | 0.1 |
| Caprylic/capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Antiseptic, pigment and fragrance | Adequate |
| Purified water | Balance |

### [Formulation Example 5] Pack

TABLE 6

| Ingredients | Contents (wt %) |
|---|---|
| Example 1, Example 2 or Example 3 | 0.001 |
| Glycerin | 4.0 |
| Polyvinyl alcohol | 15.0 |
| Hyaluronic acid | 5.0 |
| β-Glucan | 7.0 |
| Allantoin | 0.1 |
| Nonyl phenyl ether | 0.4 |
| Polysorbate 60 | 1.2 |
| Ethanol | 6.0 |
| Antiseptic, pigment and fragrance | Adequate |
| Purified water | Balance |

### [Formulation Example 6] Ointment

TABLE 7

| Ingredients | Contents (wt %) |
|---|---|
| Example 1, Example 2 or Example 3 | 0.001 |
| Glycerin | 8.0 |
| Butylene glycol | 4.0 |
| Liquid paraffin | 15.0 |
| β-Glucan | 7.0 |
| Carbomer | 0.1 |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Beeswax | 4.0 |
| Antiseptic, pigment and fragrance | Adequate |
| Purified water | Balance |

While the exemplary embodiments have been shown and described, it will be understood by those skilled in the art that various changes in form and details may be made thereto without departing from the spirit and scope of this disclosure as defined by the appended claims.

1. A method for skin moisturizing, which comprises administrating an effective amount of a composition comprising one or more of 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof as an active ingredient to a subject in need thereof.

2. A method for skin whitening, which comprises administrating an effective amount of a composition comprising one or more of 3β,23,28-trihydroxy-12-oleanene 23-caffeate, a salt thereof, an isomer thereof, a hydrate thereof or a solvate thereof; and 3β,23,28-trihydroxy-12-oleanene 33-caffeate, a salt thereof, an isomer thereof, a hydrate thereof or a solvate thereof as an active ingredient to a subject in need thereof.

3. The method according to claim 1, wherein the active ingredient is 3β,23,28-trihydroxy-12-oleanene 23-caffeate, a salt thereof, an isomer thereof, a hydrate thereof or a solvate thereof.

4. The method according to claim 1, wherein the active ingredient is 3β,23,28-trihydroxy-12-oleanene 3β-caffeate, a salt thereof, an isomer thereof, a hydrate thereof or a solvate thereof.

5. The method according to claim 1, wherein the active ingredient is comprised in an amount of 0.0001-1 wt % based on the total weight of the composition.

6. The method according to claim 1, wherein the composition comprises 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof.

7. The method according to claim 6, wherein the composition comprises 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof at a weight ratio of 1:0.5-1.5.

8. The method for skin moisturizing according to claim 1, wherein the composition further comprises a ceramide.

9. The method according to claim 8, wherein the ceramide is one or more of ceramide 1, ceramide 2, ceramide 3, ceramide 4, ceramide 5, ceramide 6, ceramide 7, ceramide 8, ceramide 102 and ceramide 104.

10. The method according to claim 1, wherein the composition is a composition for external application to skin.

11. The method according to claim 1, wherein the composition is a cosmetic composition.

12. The method according to claim 2, wherein the active ingredient is 3β,23,28-trihydroxy-12-oleanene 23-caffeate, a salt thereof, an isomer thereof, a hydrate thereof or a solvate thereof.

13. The method according to claim 2, wherein the active ingredient is 3β,23,28-trihydroxy-12-oleanene 3β-caffeate, a salt thereof, an isomer thereof, a hydrate thereof or a solvate thereof.

14. The method according to claim 2, wherein the active ingredient is comprised in an amount of 0.0001-1 wt % based on the total weight of the composition.

15. The method according to claim 2, wherein the composition comprises 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof.

16. The method according to claim 15, wherein the composition comprises 3β,23,28-trihydroxy-12-oleanene 23-caffeate, a salt thereof, an isomer thereof, a hydrate thereof or a solvate thereof; and 3β,23,28-trihydroxy-12-oleanene 3β-caffeate, a salt thereof, an isomer thereof, a hydrate thereof or a solvate thereof at a weight ratio of 1:0.5-1.5.

17. The method according to claim 2, wherein the composition is a composition for external application to skin.

18. The method according to claim 2, wherein the composition is a cosmetic composition.
